(19) United States
(12) Patent Application Publication
(10) Pub. No.: US 2025/0226085 A1
(43) Pub. Date: Jul. 10, 2025

(54) SMART CONTEXT-AWARE SEARCH AND RECOMMENDER SYSTEM FOR GUIDING SERVICE ENGINEERS DURING MAINTENANCE OF MEDICAL DEVICES

(71) Applicant: KONINKLIJKE PHILIPS N.V., EINDHOVEN (NL)

(72) Inventors: Qi GAO, EINDHOVEN (NL); Kathy Mi Young LEE, WESTFORD, MA (US); Milosh STOLIKJ, EINDHOVEN (NL); Bart Albertus Gerardus VAN DER VELDEN, EINDHOVEN (NL); Jurgen Jan RUSCH, EINDHOVEN (NL); Eugene Alekseyevich IVANOV, EINDHOVEN (NL); Cornelis Johannes Fransiscus Maria BERGMANS, ESCH (NL); Mauro BARBIERI, EINDHOVEN (NL); Louis VAN DER STAM, EINDHOVEN (NL); Raghavan RANGANATHAN, BEST (NL); Emanuele BASTIANELLI, EINDHOVEN (NL); Poovaramanan KANNAN, BANGALORE (IN); Deepak KINI, BANGALORE (IN); Pandurangaiah SIMBHOTHU, BANGALORE (IN); Ajay MUMMADISETTI, BANGALORE (IN); Krishna KUMAR, BANGALORE (IN); Sri HARSHA, BANGALORE (IN)

(21) Appl. No.: 18/700,303

(22) PCT Filed: Oct. 14, 2022
(86) PCT No.: PCT/EP2022/078715

§ 371 Date: Apr. 11, 2024

Related U.S. Application Data
(60) Provisional application No. 63/256,649, filed on Oct. 18, 2021.

(51) Int. Cl. G16H 40/40; G16H 30/00; G16H 40/67

(52) U.S. Cl. CPC G16H 40/40; G16H 30/00; G16H 40/67

(57) ABSTRACT

A non-transitory computer readable medium (107, 127) stores instructions readable and executable by at least one electronic processor (101, 113) to: retrieve, based on a query for a current service case, a plurality of the historical service case records (130) for historical service cases; rank the retrieved historical case service records to generate a ranked list (134) of historical service cases; extract service actions from the historical service case records for the ranked list of historical cases; recommend a plurality of recommended service actions for the current service case based on the service actions extracted from the historical service case records for the ranked list of historical service cases; and display, on a display device (105) of a remote electronic processing device (102) operable by a service engineer, a user interface (UI) (136) having user-selectable windows for displaying the ranked list of historical service cases and the recommended service actions.

### FIELD

The following relates generally to the medical device maintenance arts, service action recommendation arts, service action reporting arts, and related arts.

### BACKGROUND

A medical imaging system may occasionally malfunction, i.e., fail to work properly. One or multiple service engineers (SE) are then assigned to diagnose and resolve the issue. The service engineers can perform the diagnosis remotely, or on site. During diagnosis, the SE can interact with several entities that can assist him or her during system troubleshooting, and for identifying the best course of action to resolve the issue. For example, the SE may decide to: contact the customer to get a description of the problem, inspect the log files of the medical imaging system to identify relevant diagnostic information such as error messages, search through historical service records to find similar service cases and identify how they were resolved, search through technical documentation for possible root causes and potential solutions, and so forth.

After the troubleshooting process, the SE should be able to determine the root cause of the problem and identify the right service solution. The service solution can be executed remotely (e.g., a software update, remote calibration, etc.) or on-site when some spare parts need to be replaced.

SEs are often under time pressure to perform the troubleshooting and decide on the service action. Usually, SEs need to collect and examine relevant information from various sources for troubleshooting. The technical experience of SEs also differs.

The following discloses certain improvements to overcome these problems and others.

### SUMMARY

In one aspect, a non-transitory computer readable medium stores historical service case records generated from log files from one or more medical imaging devices; and instructions readable and executable by at least one electronic processor to: retrieve, based on a query received from a service engineer for a current service case, a plurality of the historical service case records for historical service cases having features in common with the current service case; rank the retrieved historical case service records to generate a ranked list of historical service cases: extract service actions from the historical service case records for the ranked list of historical cases: recommend a plurality of recommended service actions for the current service case based on the service actions extracted from the historical service case records for the ranked list of historical service cases; and display, on a display device of a remote electronic processing device operable by the service engineer, a user interface (UI) having user-selectable windows for displaying the ranked list of historical service cases and the recommended service actions.

In another aspect, a method of recommending service actions to be performed by a service engineer to service a medical imaging device includes retrieving, based on a query received from the service engineer for a current service case for the medical imaging device, a plurality of the historical service case records for historical service cases having features in common with the current service case: ranking the retrieved historical case service records to generate a ranked list of historical service cases: extracting service actions from the historical service case records for the ranked list of historical cases: recommending a plurality of recommended service actions for the current service case based on the service actions extracted from the historical service case records for the ranked list of historical service cases: displaying, on a display device of a remote electronic processing device operable by the service engineer, a UI having user-selectable windows for displaying the ranked list of historical service cases and the recommended service actions: constructing a summary of the recommended service actions and/or the list of ranked historical service cases; and displaying the summary on the UI.

In another aspect, a service device includes a display device: at least one user input device: at least one electronic processor; and a non-transitory storage medium storing instructions readable and executable by the at least one electronic processor to provide, on the display device, a UI displaying a ranked list of historical service cases and recommended service actions for a current service case for a medical imaging device.

One advantage resides in aiding a SE in determining a service action for servicing a medical device.

Another advantage resides in ranking recommended service actions for an SE to perform during a servicing case for a medical device.

Another advantage resides in ranking recommended service actions for an SE from multiple data sources.

Another advantage resides in ranking recommended service actions for an SE based on contexts of historical service cases.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for recommending at least one service action for servicing a medical device in accordance with the present disclosure.

FIG. 2 shows exemplary flow chart operations of the system of FIG. 1.

FIG. 3 shows another embodiment of the system of FIG. 1.

FIGS. 4-8 show example user interfaces generated by the system of FIG. 3.

### DETAILED DESCRIPTION

Intake of telephonic service requests from customers can typically be performed by a remote service engineer (RSE). The job of the RSE is to collect information about the imaging device malfunction telephonically, bring up the most recently uploaded log file for the system for inspection, and to ideally resolve the problem via telephone. If this is not possible, the RSE collects information to provide to a field service engineer (FSE) who will perform a site visit to resolve the problem.

A large database of historical service cases can potentially assist the RSE in performing the initial call intake. It can be beneficial to look at the previous successful cases (i.e., a problem resolved by performing a correct service action) from other SEs. However, often due to the complexity of problem and medical system as well as the amount information reported in these historical cases, and the time pressure on the SE, it is challenging for the SEs to quickly identify the most relevant cases for the current problem and system configuration. Thus, while this large database of historical cases is potentially helpful, in practice it is not feasible to go through these cases in near-real time while on the telephone with the customer.

The following discloses a system for enabling the RSE to efficiently consult the contents of the database of historical cases in near-real time. In one illustrative approach, to initiate the process, the RSE enters a search string, such as an error code recorded in the machine log at about the time the malfunction manifested. The system additionally automatically extracts information about the malfunctioning machine, such as modality and configuration. These information are used to select a ranked list of the closest historical service cases in the database. To improve the ranking, the system further weights cases by complexity and information completeness. More complex cases are ranked higher, under the expectation that simpler cases are likely to be resolved without resort to comparative historical cases. Historical cases with more complete information from both the RSE and FSE are weighted higher than cases with less complete information, based on the expectation that cases with more complete information are likely to be more helpful in resolving the current case. In some embodiments, service contract information may be used in the information completeness ranking, as historical cases processed under a more comprehensive service contract are more likely to have more complete information.

A service action recommender then processes the most similar cases to generate a ranked list of recommended service actions. Selection of the recommended service actions is based on compatibility of the service action with the configuration of the imaging device of the current service call and the fraction of the most similar cases in which that service action was performed. Other information such as cost of the service action may also be used as weightings for the ranked list of recommended service actions.

A user interface is provided, via which this information is presented. The closest historical cases and the recommended actions are presented in separate selectable windows of the UI. If the user selects a particular historical case, then additional information may be displayed for that case, as a summary or as a more detailed description. Selection of an action in the recommended service actions window will bring up a list of cases in which that action was performed. The user is also provided with a filter dialog via which the user can input specific filter terms or the like to filter the lists of closest cases and recommended actions to semi-manually drill down further.

A service report publisher component constructs a summary of the recommended actions and/or closest historical service cases. In one embodiment, the user can right-click on an action or case and select an “add to report” contextual menu option to automatically copy the information to the report. The resulting report is provided to the FSE if one is called, as an efficient hand-over of the case from the RSE to the FSE. Alternatively, if the case is resolved by the RSE without requiring a site visit then the report can serve as documentation of the resolution of the call by the RSE.

Advantageously, the foregoing processing can be performed in near-real time, e.g., within a few seconds of entering the initial search term (e.g., the error code) the lists of most similar service calls and recommended actions are populated and available to the RSE. This enables the RSE to quickly leverage the database of past service calls in resolving a current call, or in preparing information for hand-over to an FSE.

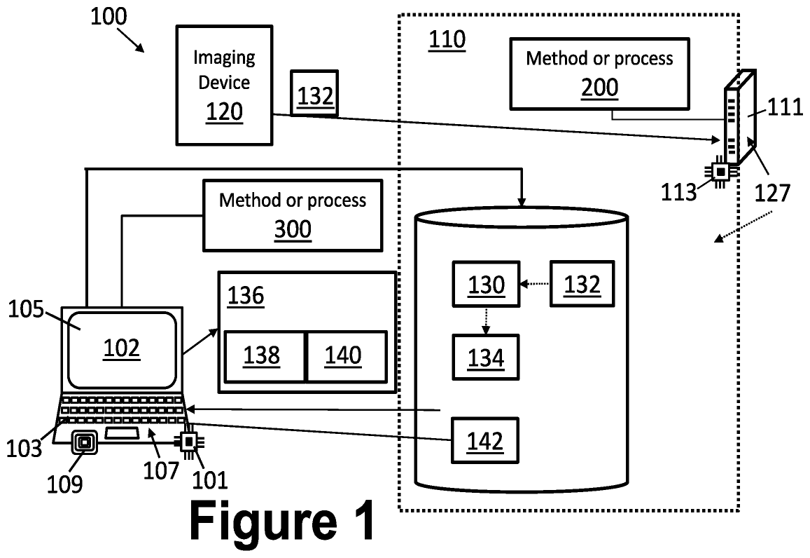

With reference to FIG. 1, an illustrative servicing support system 100 for supporting a service engineer in servicing a device 120 (e.g., a medical imaging device—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, an X-ray device, an ultrasound (US) device, or so forth. (More generally, the disclosed approach can be applied in conjunction with any type of computerized device that automatically generates log data that are analyzed by predictive models to predict component failures, e.g., the approach could be applied to a commercial airliner, radiation therapy device, or so forth). As shown in FIG. 1, the servicing support system 100 includes, or is accessible by, a service device 102 that may for example be a workstation used by a RSE. In another example, the service device 102 may be a portable device such as a notebook computer that is carried or accessed by an FSE. The service device 102 can be a desktop computer or a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g., notebook computer, tablet computer, or so forth) carried by a FSE performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer.

The service device 102 includes a display 105 via which alerts generated by predictive failure models are displayed, along with likely root cause and service action recommendation information as disclosed herein. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the servicing support system 100. The service device 102 also includes a communication interface 109 to communicate with a backend server or processing device 111, which typically implements the computational aspects of the servicing support system 100 (e.g., the server 111 has the processing power for implementing computationally complex aspects of the servicing support system 100). Such communication interfaces 109 include, for example, a wired and/or wireless Ethernet interface (e.g., in the case in which the service device 102 is a RSE workstation); or in the case in which the service device 102 is a portable FSE device the interface 109 may be a wireless Wi-Fi or 4G/5G interface or the like for connection to the Internet and/or an intranet. Some aspects of the servicing support system 100 may also be implemented by cloud processing or other remote processing (that is, the server computer 111 may be embodied as a cloud-based computing resource comprising a plurality of interconnected servers).

In illustrative FIG. 1, the servicing support system further includes a backend 110 (e.g., implemented and/or owned by the imaging device vendor or other servicing contractor, or by the medical facility that owns or leases the imaging device 120). The backend 110 receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend processing for performing predictive fault modeling and (as disclosed herein) root cause and service recommendation analyses is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the database backend 110 will receive log data from many medical imaging devices (e.g., tens, hundreds, or more imaging devices) and performs the disclosed processing for each such medical imaging device.

With continuing reference to FIG. 1, the non-transitory computer readable medium 127 stores historical service case records 130. These records may include electronic reports prepared and filed by service engineers, and data automatically generated from log files 132 from one or more medical imaging devices 120, such as current imaging device configuration information. The reports are finalized and uploaded by service engineers upon completion of respective historical service cases, and/or log files 132 can be automatically transferred from the medical imaging device(s) 120, and used by the server 111 to generate the historical service records 130. The historical service case records 130 may also include additional and/or other collected information such as summaries or (if permissible) transcripts of text, telephonic, or video communications with the customer reporting the malfunction addressed in the service call.

The non-transitory storage medium 127 stores instructions executable by the electronic processor 113 of the backend server 111 to perform a method 200 of recommending service actions to be performed by a SE to service the medical imaging device 120.

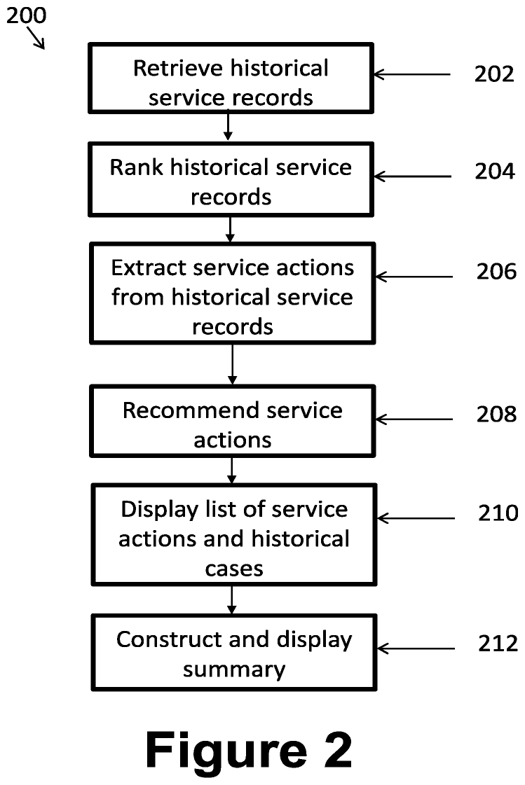

With continuing reference to FIG. 1 and further reference to FIG. 2, an illustrative embodiment of the method 200 executable by the electronic processor 113 is diagrammatically shown as a flowchart. In some examples, the method 200 may be performed at least in part by cloud processing.

To begin the method 200 of recommending service actions, the SE uses the service device 102 to input a query (via the at least one user input device 103) for a current service case in servicing the medical imaging device 120. In some embodiments, the query comprises an error code stored in the log files 32 of a current medical imaging device 120 of the current service case. At an operation 202, a plurality of historical service case records 130 are retrieved from the non-transitory computer readable medium 127 for historical service cases having features in common with the current service case. To do so, the retrieving operation 102 includes extracting, from the historical service cases 130, features of the historical service cases 130, including at least an imaging modality and a configuration of the current medical imaging device 120. The features in common with the current service case include the imaging modality and at least a portion of the configuration.

At an operation 204, the retrieved historical case service records 130 are ranked to generate a ranked list 134 of historical service cases 130. The ranking can be based on factors such as the similarity of the imaging devices involved in the historical service cases 130 to the imaging device 120 of the current service case and/or relationship of the query to the historical service cases 130. Additionally, one or more “usefulness” and/or “quality” metrics may be computed for the historical service cases 130 and used in the ranking. In some embodiments, for example, the ranking operation 204 can include generating complexity metrics for the historical service records 130 based on complexity levels of the historical service cases 130 recorded in the historical services records 130, and also generating completeness metrics for the historical service records 130 based on completeness levels of the historical service records 130. The complexity metrics are useful for assessing usefulness of the historical cases, since the SE is more likely to be consulting historical service cases when attempting to resolve a complex current service case that is not easy to resolve-hence, more complex historical service cases are likely to be more useful. The completeness metrics are useful in assessing quality of the historical information-even if a historical case is very relevant, if the amount of information on that case is incomplete or limited then it may be of limited usefulness to the SE. Weights can then be obtained for the historical service records 130 based on the generated similarity metrics, complexity metrics and/or completeness metrics and/or optionally other ranking criteria. The historical service records 130 can then be ranked based on scores for the historical service records weighted by the respective complexity metrics and completeness metrics. The ranking 204 thus identifies a relatively small subset of the historical service cases 130 in the non-transitory computer readable medium 127 that are most likely to be useful in resolving the current service case.

At an operation 206, service actions from the historical service case records 130 can be extracted for the ranked list 134 of historical service records 130. At an operation 208, a plurality of recommended service actions can be recommended based on the service actions extracted from the historical service case records 130 for the ranked list 134 of historical service cases 130. In some embodiments, the recommending operation 208 includes recommending the plurality of recommended service actions based on a compatibility of each recommended service action with a configuration of the current medical imaging device 120. In other embodiments, the recommending operation 208 includes recommending the plurality of recommended service actions based on a fraction of most similar historical services records 130 in which each recommended service action was performed. Such factors can also be combined in determining which service actions to recommend.

At an operation 210, a user interface (UI) 136 is displayed on the service device 102 (or another remote electronic processing device operable by a SE) in which the UI 136 displays the ranked list 134 of historical service cases 130, and the recommended service actions. In some embodiments, as shown in FIG. 1, a first user selectable window 138 can be displayed in the UI 136 of the ranked list 134 of historical service cases 130, and a second user selectable window 140 can also be displayed in the UI 136. The UI 136 is operable by the SE to selectively display one or both of the first selectable window 138 and the second selectable window 140 on the display device 105.

In some embodiments, the SE can interact with the UI 136 via the at least one user input device 103. In one example, the SE can use the at least one user input device 103 to select one of the historical service cases 130 in the displayed ranked list 134. The UI 136 can then display a window presenting a description of the selected historical case 130. In another example, the SE can select one of the display service actions in the ranked list 134, and the UI 136 can then display a window presenting a list of historical service cases 130 in which the selected service action was performed. In a further example, a graphical UI (GUI) dialog with fields can be displayed on the UI 136, thereby allowing the SE to input filter terms via the at least one user input device 103 of the service device 102. The SE can then select one or more of the filter terms, and historical service cases 130 having the filter terms can be displayed on the UI 136.

In an optional embodiment 212, a summary 142 of the recommended service actions and/or the list 134 of ranked historical service cases 130 can be displayed on the UI 136. The SE can then select one of the recommended service actions and/or one ranked historical service cases 130 in the summary 142. The selected service action and/or the historical service case 130 can then be copied to generate a current service case report, which can be transmitted from, for example, a remote electronic processing device (e.g., a workstation used by a RSE) to the service device 102 operated by an FSE performing the current service case on the medical imaging device 120.

### Examples

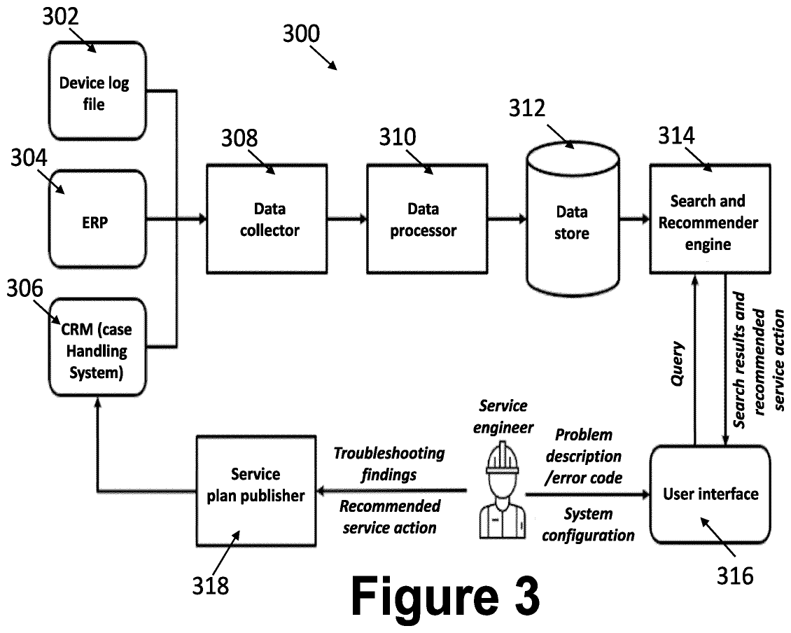

The following describes examples of the servicing support system 100. FIG. 3 shows another embodiment of servicing support system 300. As shown in FIG. 3, the servicing support system 100 retrieves data from multiple sources, including a device log file 302 of the medical imaging device 120 (see FIG. 1), an Enterprise Resource Planning (ERP) system 304 for managing contracts, and a customer relation management (CRM) system 306 for service case handling.

FIG. 3 also shows several modules implemented in the server 111. As shown in FIG. 3, a data collector 308 is configured to retrieve various data elements from multiple source systems (e.g., the device log file 302, the ERP system 304, and the CRM system 306). The retrieved data elements can include, for example, historical service work orders (a service work order consists of a problem description from the customer, case activities which contains the troubleshooting findings and repair actions recorded by service engineers. The case activities may be recorded by either remote service engineers who perform the troubleshooting remotely in the call centre or field service engineers who travel to the customer's site to repair the system on-site): system configuration details of the medical device 120 (e.g., product type, serial number of the system, and software release): information on spare parts (e.g., a unique identifier of a part, textual description, cost (price) and images of the part, and so forth); and information on service contracts.

The retrieved data elements may be in a format of free text entered by a human (e.g., the case activity reports written by SEs) or in a more structured format (e.g., data retrieved from other databases). A data processor 310 is configured to process all the data elements retrieved by the data collector 308, extract the relevant information and also establish relationships between different elements. The output of the data processor 310 is a set of data objects (so-called service cases) which contains structured data on historical service work orders including all information gather from the various data sources described above. These data objects are stored in a data store 312, which can be used by a search engine for fast querying.

A search and recommender engine 314 is used to search the historical service cases stored in the data store 312 to find the most relevant service cases given a search query. The search and recommender engine 314 further ranks the relevant service cases based on a relevance score, which can be calculated from a number of factors including a similarity between a historical case and a given query, a complexity of a historical case which indicates how difficult the problem of the system was, and a completeness of a historical case, which indicates how detailed the troubleshooting and repair activities were recorded. A SE can formulate a search query in multiple manners. One way is by entering a textual description of a problem, based on information that the SE received from the customer or operator of the medical device 120. Another way is by entering a message found in the log files 302 created by the medical device 120 under maintenance. These log files 302 may contain critical information about the root cause of the problem. In addition to the problem description, the SE can also provide system configuration properties as additional search criteria.

The search and recommender engine 314 further ranks the relevant service cases by calculating a relevance score in the following manner. Let a system, or machine, be denoted by m, and the configuration of the machine is denoted by sₘ. A query entered by a SE is denoted by q. A service case is denoted by c, a set of cases is denoted by C. The search and recommender engine 314 performs the following steps to return a list of ranked relevant cases, denoted by C(q). The original query entered by the SE is converted to a term vector \vec{t_{q}} representing the textual problem description and if applicable S_{q} representing the machine configuration.

For each case cᵢ in C, a score is calculated according to Equation 1:

$$score ( \vec{{t}_{q}} , {c}_{i} ) = p ( {s}_{q} , {s}_{{c}_{i}} ) s im ( \vec{{t}_{q}} , {c}_{i} ) \qquad ( 1 )$$

where p(s_{q}, s_{c}_{ᵢ}) is a function that determines if the entered system configuration is compatible with the system configuration of cᵢ, denoted by s_{c}_{ᵢ}:

$$p ( {s}_{q} , {s}_{{c}_{i}} ) = \{ \begin{matrix}0 , {s}_{q} is not compatible with {s}_{{c}_{i}} , \\ 1 , {s}_{q} is not compatible with {s}_{{c}_{i}}\end{matrix} .$$

and sim (\vec{t_{q}}, cᵢ) is a function that determine the similarity between the term vector \vec{t_{q}} and the case cᵢ, with a range of [0, 1].

The search and recommender engine 314 returns a set of cases C(q), in which the score of each case is greater than a pre-defined threshold θ, i.e.: for any case cᵢ∈C(q), score (\vec{t_{q}}, cᵢ)>θ.

When displaying the returned cases C(q) to the SE, the cases are ranked by taking a number of factors into consideration. For any ∈C(q), its rank is calculated according to Equation 2:

$$rank ( {c}_{i} ) = ( \vec{{t}_{q}} , {c}_{i} ) + complexity ( {c}_{i} ) + completeness ( {c}_{i} ) \qquad ( 2 )$$

where score (\vec{t_{q}}, cᵢ) is calculated as in Equation 1: complexity (cᵢ) is a function that determines
the complexity of the case cᵢ according to:

$$complexity ( {c}_{i} ) = \{ \begin{matrix}1 , & if {c}_{i} is a simple case \\ 2 , & if {c}_{i} is a case with medium complexity \\ 3 , & if {c}_{i} is a complex case\end{matrix}$$

completeness (cᵢ) is a function that determines the completeness of the case cᵢ, i.e.: to what extent all relevant troubleshooting and repair activities are correctly and fully reported for cᵢ.

$$completeness ( {c}_{i} ) = \{ \begin{matrix}1 & if Condition 1 is met \\ 2 & if Condition 2 is met \\ 3 & if Condition 3 is met\end{matrix}$$

where: Condition 1 is met if the historical service case cᵢ contains incomplete information reported by both remote and field service engineers: Condition 2 is met if cᵢ contains complete information reported by only remote or field service engineer; and Condition 3 is met if cᵢ contains complete information reported by both remote and field service engineers.

More generally, the ranking for each historical service case (cᵢ) may be calculated based on an aggregation of a score (\vec{t_{q}}, cᵢ) metric, a complexity (cᵢ) metric, and a completeness (cᵢ) metric, where q denotes the query received from the service engineer for the current service case and score (\vec{t_{q}}, cᵢ) measures similarity of the query q and the historical service case cᵢ and complexity (cᵢ) quantifies a complexity of the historical service case cᵢ and completeness (cᵢ) quantifies a completeness of services records of the historical service case cᵢ.

Advantageously, the search and recommender engine 314 provides a collection of relevant historical cases with similar problems to the current case which the SE is handling. By taking consideration of various factors including similarity, complexity and completeness when ranking these cases, the SE can more easily and quickly get useful information on troubleshooting and repair actions from the top ranked cases.

The search and recommender engine 314 is also configured to recommend service actions for the current service case. To do so, several inputs, such as a configuration of a malfunctioning device 120 (i.e., system configuration) and a set of related historical service records 130, along with corresponding service actions performed for the historical service records 130, are input to the search and recommender engine 314. Using these inputs, the search and recommender engine 314 is configured to output one or more service actions which may resolve the problem which the SE is working on. Internally, the search and recommender engine 314 can consult an external database (not shown) to determine whether a service action is compatible for the malfunctioning device 120, and to determine a compatible (i.e., alternative) service action for the given malfunctioning device 120.

The search and recommender engine 314 further recommends the service actions by calculating a recommendation score in the following manner. Let a system, or machine, be denoted by m. A service case is denoted by c, a set of cases is denoted by C, and the set of cases for a machine mᵢ is denoted by C(mᵢ). A service action is denoted by a, a set of actions is denoted by A, and the set of service actions performed in service case cᵢ is denoted by A (cᵢ). Let compatible be a function that determines whether a service action is compatible with a given machine, according to:

$$compatible ( a , m ) = \{ \begin{matrix}0 , & a is not compatible for m , \\ 1 , & a is compatible for m\end{matrix} .$$

Let equivalent be a function that, given a service action a, and machine m, for which that service action is not applicable (i.e. compatible (a, m)=0), returns a service action â, which has the same function as service action s, but is suitable for machine m (i.e. compatible (â, m)=1). If no such service action â exists, then the equivalent function returns a pre-defined symbol indicating an empty list (e.g., Ø). Obviously, for a given service action s, if compatible (a, m)=1, then equivalent (a, m)=a.

If the SE is working on a service case cⱼ, for machine mⱼ, then the search and recommender engine 314 is configured to calculate a recommendation score according to Equation 3:

$$A = {⋃}_{{c}_{i} ∈ C} A ( {c}_{i} ) \qquad ( 3 )$$

where C is a set of related service actions, and A is a set of service actions. A new set of service actions is created, where every service action in A is replaced with a compatible service action for machine mⱼ, provided that such a service action exists, according to Equation 4:

$$\hat{A} = \{ equivalent ( a , {m}_{j} ) \vert ∀ a ∈ A , equivalent ( a , {m}_{j} ) \neq ∅ \} \qquad ( 4 )$$

For every service action â in Â, its rank is calculated, defined as fraction of service cases in C that have used a compatible service action, according to Equation 5:

$$rank ( \hat{a} ) = \frac{\vert \{ c ∈ C \vert ∃ a ∈ A ( c ) : equivalent ( a , {m}_{j} ) = \hat{a} \} \vert}{\vert C \vert} \qquad ( 5 )$$

The set of service actions A is sorted in descending order by their appropriate rank. If two service actions have the same rank, then they are ordered based on their complexity or price (if available), in descending order.

The first k items from the ordered set of service actions Â is shown as recommended service actions, where k is a pre-defined constant (e.g., 4). Alternatively, a more complicated criterion can be used, such as recommending as many items as possible from Â such that a pre-defined percentage of the service cases in C are covered, or such that the total price of the recommended service actions should be below a pre-defined threshold.

The rank of the suggestions is visually presented to the SE on the service device 102, in order to help the decision-making process.

Advantageously, the search and recommender engine 314 provides actionable insights to the SE. The recommendations of service actions are based on the usage of service actions for resolving similar service records in the past, which should result in more accurate recommended suggestions. In addition, combinations of service actions can be ranked, rather than individual actions, and can include historical service records of the malfunctioning device 120 in the service action recommendation.

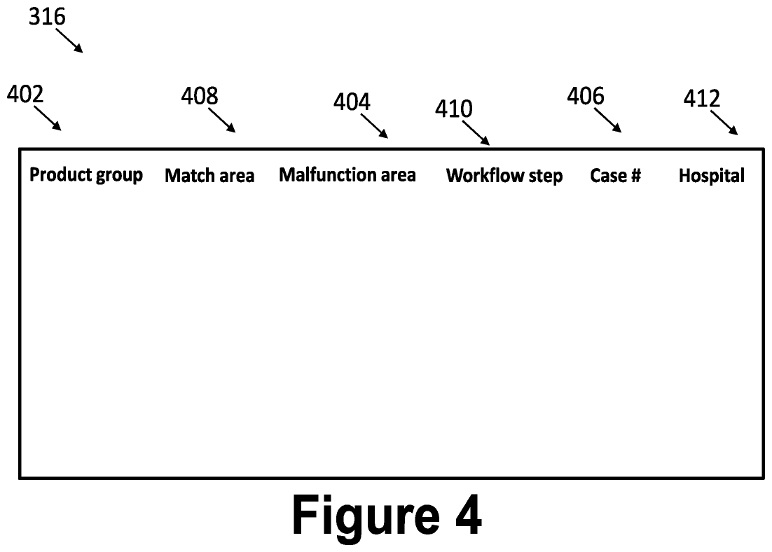

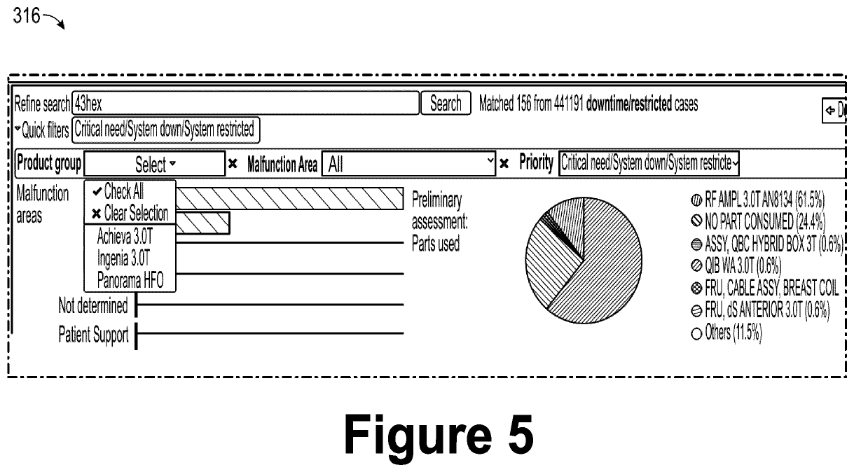

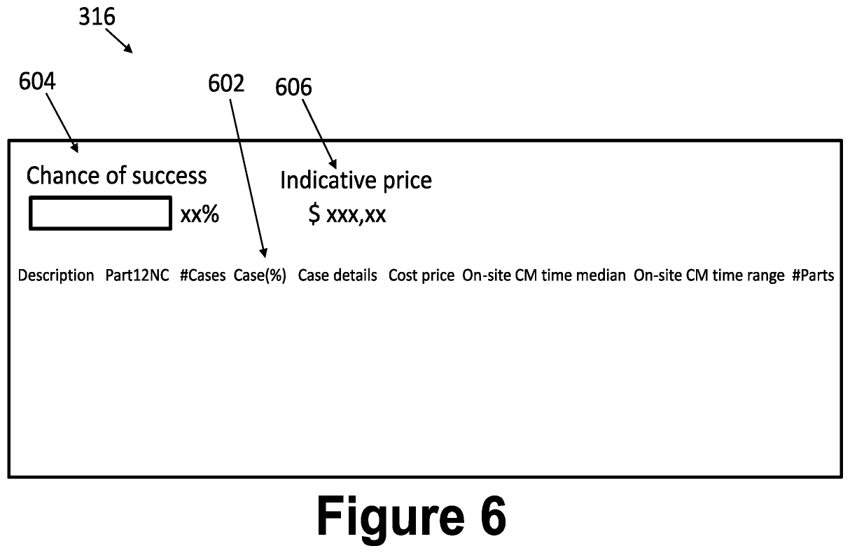

FIG. 3 also shows an example of a UI 316 (corresponding to the UI 136 in FIG. 1). The UI 316 allows a SE to browse the relevant historical cases and service actions output by the search and recommender engine 314. The UI 316 allows the SE to enter a problem description, an error code, and/or a system configuration as a search query. The UI 316 also allows the SE to view details of the relevant cases from the search and recommender engine 314. FIG. 4 shows an example of the UI 316, which shows critical attributes of a case are displayed in a table grid with fields including a subject field (i.e., short description of problem), a product group field 402, a malfunction area field 404 (i.e., a subsystem where the problem occurred), a case number field 406, customer name field, etc. Furthermore, a summary which contain pieces of short text snippets indicating where the query appears in the textual description of various data attributes of a case, a so-called match area field 408. A workflow step field 410 (indicative of a workflow step when the malfunction took place) and a hospital field 412 (where the medical device 120 is located) are also included. Error! Reference source not found. shows an example of this feature.

The UI 316 also allows the SE to filter relevant cases by selecting a relevant system configuration, and a malfunction area where a problem could occur. The filtering process also dynamically changes the search results (i.e., relevant cases) as well as the recommended service actions. Error! Reference source not found. shows an example of this feature.

The UI 316 also provides other visualization features for the SE. Such features can include detailed information on the recommended service actions, including: a spare part descriptions and identification field 602, information on parts ordering (e.g., prices, whether a review is required before purchasing, and so forth), statistics calculated based on relevant historical cases (which indicates the amount of time the SE would need to replace the corresponding spare part when on-site), a probability of selected service actions for fixing the current problem, a “success chance” filed 604, an estimated cost filed 606, and so forth. Error! Reference source not found. shows an example of this feature.

Advantageously, the UI 316 provides a user-friendly interface which provides the most critical information which is required for SEs to reviewing the case search results and deciding on the most likely service solutions to repair the current problem.

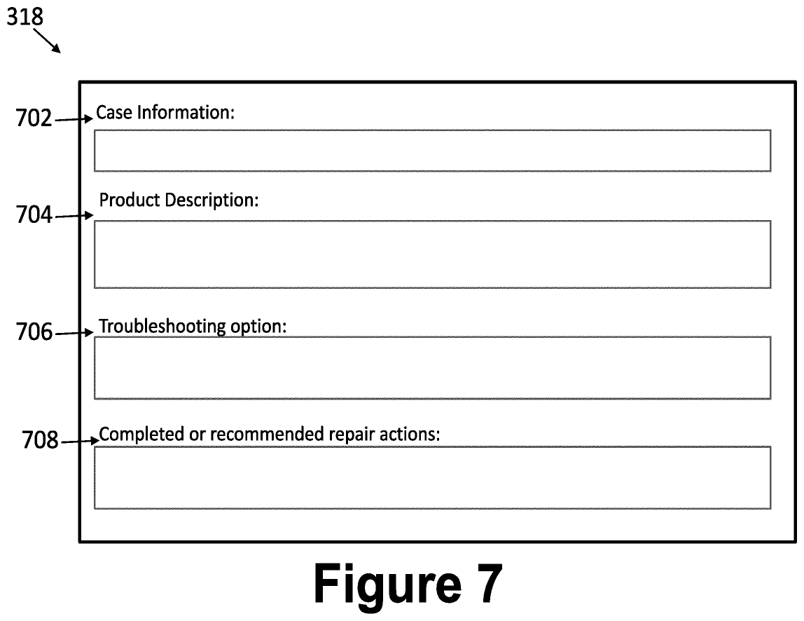

Referring back to FIG. 3, the servicing support system 300 also includes a service plan publisher 318 that assists a SE to record the main troubleshooting findings and recommended service actions in a structured format and publish the content back to the case handling system automatically. When the SE finishes the troubleshooting with the decision on the service action, the main findings are required to be reported, as well as the action, and upload the report to the case handling system. The system 300 provides an interface for the service plan publisher 318 that assists SE to report the essential findings in a structured format. FIG. 7 shows an example interface of the service plan publisher 318. The findings are grouped and reported in four sections with corresponding fields including a case information field 702, a problem description filed 704, a troubleshooting options field 706, and a completed or recommended repair action filed 708. For each field, there are several attributes where the SE can enter the corresponding information. After the SE has filled in all the required fields, the textual report is automatically generated and automatically uploaded to the case handling system.

Advantageously, the service plan publisher 318 helps the SE report the findings more efficiently. With the automatically generated service report with a fixed template, it reduces the time needed for the SE to finish the service report as well as it improves the quality of the service report. With more such service reports with structured format uploaded to the case handling system over time, it will also improve the quality of case search and recommended service actions.

The case summary generated by the service plan publisher 318 provides ways of displaying case information that help the SE understand the service issue. The case summary can include, for example, details about the product and the facility where it is used, a defect as reported by the customer, diagnostic steps as performed by SEs, solution details, remarks made by SEs on the process, a list of parts which were used to solve the malfunction, and so forth. Diagnostic steps and solutions are performed, suggested, implemented, and tested by RSEs, who are located at a call center, and FSEs who visit the facility.

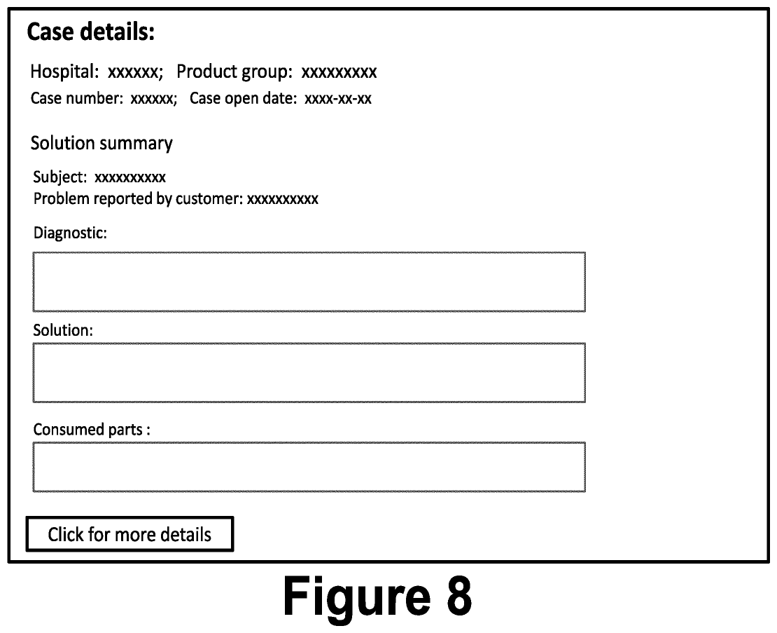

The diagnostic steps and solution steps may involve multiple (i.e., virtual) sessions at the facility where the product is located. SEs may add remarks for their colleagues or reminders to themselves. The reports from those sessions are combined into a section for diagnostics and a section for the solution. Both sections are divided into activities performed remotely (i.e., by a RSE) and activities performed onsite (i.e., by a FSE). FIG. 8 shows an example of a case summary. The same information as listed before may also presented in a fully chronological method. In this presentation every diagnostic finding, remark, proposed solution, part replacement, test etc. is shown in a list. Every item in the list shows the date/time, the time since the start of the case and all details. This list may also contain more detail than the initial summary.

Advantageously, the case summary provides insights to the SE that help determine whether a historical case if sufficiently similar to the issue they are currently trying to solve. The summary enables the SE to see at a glance whether the historical case is relevant. This saves time. The detailed view allows the SE to dig deeper to see whether the process of the historical case contains additional clues on how to solve the current issue.

In a particular implementation example, the system 300 can be implemented in a Case History Analyzer (CHA) application, of a customer who has a maintenance contract with a vendor for the imaging device. The customer typically calls when the machine fails to function correctly. In the call the SE creates a ‘case’ for this call. The case is then ‘opened’. The SE must diagnose the problem and must decide on a service activity. This service activity can be an action that can be executed by the customer, e.g., reboot the system but can also be the dispatching of a FSE to replace specific parts in the machine at hand. As soon as the machine functions according to specifications again the case is considered ‘solved’ and is then closed.

For any case one can look, in retrospect, whether it has been solved remotely (e.g., by the advice to reboot the machine) or whether it has been solved with exactly one visit by a remote service engineer in which one or more parts were replaced. In these two cases the term ‘first time right’ (FTR) applies. In all other cases, i.e., where two or more visits by a RSE were required to close the case, the term ‘first time right’ does not apply. The customer will be happy when a case has been solved ‘first time right’. When not solved ‘first time right’ higher costs are involved because the field service engineer must travel to the customer more than once and because more parts are replaced during these multiple visits.

During a customer call the SE will typically find a phrase that best describes the problems reported by the customer. This can be an error message observed by the customer, a log line from a log file extracted from the machine etc. The SE then enters this phrase into the CHA which will search this phrase in all historical (closed) cases in which a problem was solved for a machine with the same characteristics as the customer's machine (typically per modality such as MR. CT, etc.). The CHA then finds all historical cases of which the description, solution or any other available data matches the entered phrase. All these cases are ranked according to several criteria and shown to the SE. The CHA also gathers statistics on all replaced parts used in the matched historical cases and presents the service engineer with a distribution of the most used parts (where ‘no parts used’ is also considered a ‘part’). Typically, this distribution is shown as a pie-chart.

Based on the presented historical cases and presented replaced parts distribution the SE creates a service order in which a set of parts to replace is output.

As used herein, the term “service action” can refer to performing some test, calibrating a subsystem, lubricating, or cleaning some part(s), et cetera. It may also involve the replacement of a part and subsequent test whether or not this solved the issue. Note that the duration of replacing a part will greatly depend on whether or not the FSE currently has a spare example of this part. If not, then replacing might cost one or more days to order and deliver the spare part. Advantageously, the service action recommendation provides information by which the FSE can bring the likely spare part, thereby avoiding this potential delay.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium: a hard disk drive, RAID array, or other magnetic disk storage media: an optical disk or other optical storage media: or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
historical service case records generated from log files from one or more medical imaging devices;
instructions readable and executable by at least one electronic processor to:
retrieve, based on a query received from a service engineer for a current service case, a plurality of the historical service case records for historical service cases having features in common with the current service case;
rank the retrieved historical case service records to generate a ranked list of historical service cases;
extract service actions from the historical service case records for the ranked list of historical cases;
recommend a plurality of recommended service actions for the current service case based on the service actions extracted from the historical service case records for the ranked list of historical service cases; and
display, on a display device of a remote electronic processing device operable by the service engineer, a user interface (UI) having user-selectable windows for displaying the ranked list of historical service cases and the recommended service actions.

2. The non-transitory computer readable medium of claim 1, wherein the query comprises an error code stored in the log files of a current medical imaging device of the current service case.

3. The non-transitory computer readable medium of claim 2, wherein the retrieving further includes:
extracting, from the historical service cases at least an imaging modality and a configuration of the current medical imaging device, wherein the features in common with the current service case include the imaging modality and at least a portion of the configuration.

4. The non-transitory computer readable medium of claim 1, wherein the ranking includes:
generating complexity metrics for the historical service records based on complexity levels of the historical service cases recorded in the historical services records;
generating completeness metrics for the historical service records based on completeness levels of the historical services records;
obtaining weights for the historical service records based on the complexity metrics and completeness metrics of the historical service cases; and
ranking the historical service records based on scores for the historical service records weighted by the respective complexity metrics and completeness metrics.

5. The non-transitory computer readable medium of claim 2, wherein the recommending includes:
recommending the plurality of recommended service actions based on a compatibility of each recommended service action with a configuration of the current medical imaging device.

6. The non-transitory computer readable medium of claim 2, wherein the recommending includes:
recommending the plurality of recommended service actions based on a fraction of most similar historical record cases in which each recommended service action was performed.

7. The non-transitory computer readable medium of claim 1, wherein the displaying of the UI includes:
displaying a first user selectable window in the UI of the ranked list of historical service cases; and
displaying a second user selectable window in the UI of the recommended service actions;
wherein the UI is operable by the service engineer to selectively display one or both of the first selectable window and the second selectable window on the display device.

8. The non-transitory computer readable medium of claim 7, wherein the instructions are further readable and executable by the at least one electronic processor to:
receive, via at least one user input device of the service device, a selection on the UI of one of the historical service cases in the ranked list; and
display, on the UI, a window presenting a description of the selected historical case.

9. The non-transitory computer readable medium of claim 7, wherein the instructions are further readable and executable by the at least one electronic processor to:
receive, via at least one user input device of a remote electronic processing device, a selection on the UI of one of the recommended service actions; and
display, on the UI, a window presenting a list of historical service cases in which the selected service action was performed.

10. The non-transitory computer readable medium of claim 7, wherein the instructions are further readable and executable by the at least one electronic processor to:
display, on the UI, a graphical user interface (GUI) dialog with fields allowing the service engineer to input filter terms via the at least one user input device of the remote electronic processing device;
receive, via the at least one user input device, a selection of one or more of the filter terms; and
display, on the UI, the historical service cases having the filter terms.

11. The non-transitory computer readable medium of claim 1, further storing instructions readable and executable by at least one electronic processor to:
construct a summary of the recommended service actions and/or the list of ranked historical service cases; and
display the summary on the UI.

12. The non-transitory computer readable medium of claim 11, wherein the instructions further include:
receive, via the at least one user input device of the remote electronic processing device, a selection of one of the recommended service actions and/or one ranked historical service cases;
copy the selected service action and/or the historical service case to generate a current service case report; and
transmit the current service case report from the remote electronic processing device to a service device operable by a field service engineer (FSE) performing the current service case.

13. The non-transitory computer readable medium of claim 1, wherein the ranking for each historical service case (cᵢ) is calculated based on an aggregation of a score (\vec{t_{q}}, cᵢ) metric, a complexity (cᵢ) metric, and a completeness (cᵢ) metric, where q denotes the query received from the service engineer for the current service case and score (\vec{t_{q}},cᵢ) measures similarity of the query q and the historical service case c; and complexity (cᵢ) quantifies a complexity of the historical service case cᵢ and completeness (cᵢ) quantifies a completeness of services records of the historical service case cᵢ.

14. A method of recommending service actions to be performed by a service engineer to service a medical imaging device, the method comprising:
retrieving, based on a query received from the service engineer for a current service case for the medical imaging device, a plurality of the historical service case records for historical service cases having features in common with the current service case;
ranking the retrieved historical case service records to generate a ranked list of historical service cases;
extracting service actions from the historical service case records for the ranked list of historical cases;
recommending a plurality of recommended service actions for the current service case based on the service actions extracted from the historical service case records for the ranked list of historical service cases;
displaying, on a display device of a remote electronic processing device operable by the service engineer, a user interface (UI) having user-selectable windows for displaying the ranked list of historical service cases and the recommended service actions;
constructing a summary of the recommended service actions and/or the list of ranked historical service cases; and
displaying the summary on the UI.

15. The method of claim 14, further including:
receiving, via the at least one user input device of the remote electronic processing device, a selection of one of the recommended service actions and/or one ranked historical service cases;
copying the selected service action and/or the historical service case to generate a current service case report; and
transmitting the current service case report from the remote electronic processing device to a service device operable by a field service engineer (FSE) performing the current service case.

16. The method of claim 14, wherein the query comprises an error code stored in the log files of a current medical imaging device of the current service case, and the retrieving further includes:
extracting, from the historical service cases at least an imaging modality and a configuration of the current medical imaging device, wherein the features in common with the current service case include the imaging modality and at least a portion of the configuration.

17. The method of claim 14, wherein the ranking includes:
generating complexity metrics for the historical service records based on complexity levels of the historical service cases recorded in the historical services records;
generating completeness metrics for the historical service records based on completeness levels of the historical services records;
obtaining weights for the historical service records based on the complexity metrics and completeness metrics of the historical service cases; and
ranking the historical service records based on scores for the historical service records weighted by the respective complexity metrics and completeness metrics.

18. The method of claim 14, wherein the recommending includes at least one of:
recommending the plurality of recommended service actions based on a compatibility of each recommended service action with a configuration of the current medical imaging device; and
recommending the plurality of recommended service actions based on a fraction of most similar historical record cases in which each recommended service action was performed.

19. The method of claim 14, wherein the displaying of the UI includes:
displaying a first user selectable window in the UI of the ranked list of historical service cases; and
displaying a second user selectable window in the UI of the recommended service actions;
wherein the UI is operable by the service engineer to selectively display one or both of the first selectable window and the second selectable window on the display device.

20. A service device, comprising:
a display device;
at least one user input device; and
at least one electronic processor;
a non-transitory storage medium storing instructions readable and executable by the at least one electronic processor to:
provide, on the display device, a user interface (UI) displaying a ranked list of historical service cases and recommended service actions for a current service case for a medical imaging device.
